(19) United States
(12) Patent Application Publication
(10) Pub. No.: US 2004/0043434 A1
(43) Pub. Date: Mar. 4, 2004

(54) Shp-2 tyrosine phosphatase and embryonic stem cell differentiation

(76) Inventor: Gen-Sheng Feng, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/460,577

(22) Filed: Jun. 12, 2003

Related U.S. Application Data
(60) Provisional application No. 60/389,275, filed on Jun. 13, 2002.

(51) Int. Cl. G01N 33/53; G01N 33/567; C12N 5/08

(52) U.S. Cl. 435/7.2; 435/366

(57) ABSTRACT

The invention relates to modulation of Shp-2 tyrosine phosphatase activity within embryonic, and likely hematopoietic, stem cells to modulate stem cell self-renewal, survival and differentiation. The invention further relates to development of Shp-2-inhibitory molecules for use in culture for ex vivo expansion of stem cells.

### RELATED APPLICATIONS

This application claims the benefit of priority under 35 U.S.C. 119 (e) of the U.S. Provisional Application No. 60/389,275, filed Jun. 13, 2002, the disclosure of which is incorporated herein by reference in its entirety.

### GOVERNMENTAL INTEREST

This invention was made with United States Government support under grant numbers GM53660 and CA78606 awarded by the National Institutes of Health. The U.S. Government has certain rights in this invention.

### FIELD OF THE INVENTION

The invention relates to modulation of Shp-2 tyrosine phosphatase activity within embryonic, and preferably hematopoietic, stem cells to modulate stem cell self-renewal, survival and differentiation. The invention further relates to Shp-2-inhibitory molecules for use in culture for ex vivo expansion of stem cells.

### BACKGROUND OF THE INVENTION

Murine embryonic stem (ES) cells are pluripotent cells with the capacity to self-renew and to differentiate into all tissues of the adult mouse, including germ cells (Nagy, A. et al. 1993 PNAS USA 90:8424-8428; Rossant, J. et al. 1993 Philos Trans R Soc Lond B Biol Sci 339:207-215). In suspension or semi-solid in vitro culture, ES cells grow into cellular spheres termed embryoid bodies (EBs). Within this cellular context, these cells have the capacity to differentiate into blood cells, muscle cells, endothelial cells, and neurons (Keller, G. M. 1995 Curr Opin Cell Biol 7:862-869; Wang, R. et al. 1992 Development 114:303-316; Wiles, M. V. 1993 Methods Enzymol 225:900-918; O'Shea, K. S. 1999 Anat Rec 257:32-41), and therefore, provide a useful reagent for the study of specific gene products in ES cell function. Stem cells have a limited repertoire of activities including differentiation to a committed cell type, self-renewal to identically duplicate itself, or programmed cell death (Eaves, C. et al. 1999 Ann N Y Acad Sci 872:1-8). The molecular mechanisms that dictate a stem cell's fate are largely unknown, but are being sought actively as stem cells are potential future pharmaceuticals for diseases such as type 1 diabetes mellitus, neuronal degeneration disorders, and hematopoietic failure disorders.

Mutant ES cells have been reported that bear a deletion in the Shp-2 tyrosine phosphatase locus resulting in an in-frame deletion of amino acids 46 to 110 within the N-terminal SH2 domain of a mature Shp-2 protein (Saxton, T. M. et al. 1997 EMBO J 16:2352-2364). Shp-2^{-/-} ES cells have a dramatically decreased capacity to differentiate into erythroid and myeloid progenitors in vitro (Qu, C. K. et al. 1997 Mol Cell Biol 17:5499-5507) and in vivo (Qu, C. K. et al. 1998 Mol Cell Biol 18:6075-6082). Additionally, a requirement of Shp-2 for T and B lymphopoiesis was demonstrated using the Rag-2 complementation system (Qu, C. K. et al. 2001 Blood 97:911-914). However, although heterozygous mice bearing the Shp-2 mutant allele appear normal, suggesting that the persistent truncated protein does not act in a dominant negative fashion (Saxton, T. M. et al. 1997 EMBO J 16:2352-2364).

Shp-2 is a ubiquitously expressed non-transmembrane tyrosine phosphatase with two SH2 domains. Shp-2 is known to be a component of several cell surface receptor-stimulated signal transduction pathways including those of epidermal growth factor, platelet-derived growth factor, stem cell factor, erythropoietin, interferon-α and -γ and leukemia inhibitory factor (LIF) (Tauchi, T. et al. 1994 J Biol Chem 269:25206-25211; Tauchi, T. et al. 1995 J Biol Chem 270:5631-5635; Bennett, A. M. et al. 1996 Mol Cell Biol 16:1189-1202; Klinghoffer, R. A. & Kazlauskas, A. 1995 J Biol Chem 270:22208-22217; Qu, C. K. et al. 1999 PNAS USA 96:8528-8533; Shi, Z. Q. et al. 1998 J Biol Chem 273:4904-4908; You, M. et al. 1999 Mol Cell Biol 19:2416-2424; Burdon, T. et al. 1999 Cells Tissues Organs 165:131-143). The role of Shp-2 in LIF-stimulated signal transduction pathways is of particular interest as ES cells are routinely cultured in high concentrations of LIF to maintain an undifferentiated, self-renewing state (Williams, R. L. et al. 1988 Nature 336:684-687). LIF signals through gp130, the common subunit for the IL-6 family of cytokines (including IL-6, IL-11, cardiotrophin-1, ciliary neurotrophic factor, and oncostatin M) (Taga, T. & Kishimoto, T. 1997 Annu Rev Immunol 15:797-819). LIF binds the heterodimeric LIF receptor-gp130 complex resulting in the activation of the Jak kinases with subsequent recruitment and phosphorylation of Shp-2 and Stat3 (signal transducer and activator of transcription 3) (Matsuda, T. et al. 1999 EMBO J 18:4261-4269). The Jak-Stat pathway, in general, has been found to be important for stem cell self-renewal in Drosophila (Tulina, N. & Matunis, E. 2001 Science 294:2546-2549; Kiger, A. A. et al. 2001 Science 294:2542-2545). Specifically, activated Stat3 is known to be involved in the maintenance of mammalian ES cell self-renewal (Burdon, T. et al. 1999 Cells Tissues Organs 165:131-143; Niwa, H. et al. 1998 Genes Dev 12:2048-2060) and has been shown to upregulate pro-survival molecules resulting in decreased apoptosis (Epling-Burnette, P. K. et al. 2001 J Clin Invest 107:351-362; Catlett-Falcone, R. et al. 1999 Immunity 10:105-115). The functions of self-renewal and apoptosis are closely integrated in determining stem cell fate. For example, neural stem cells lacking the tumor suppressor gene Pten have decreased apoptosis as well as increased cell proliferation resulting in an increased number of total cells within the Pten^{-/-} brain (Groszer, M. et al. 2001 Science 294:2186-2189).

ES cells genetically modified to express a G-CSF-gp130 chimeric receptor bearing a mutation at the Shp-2 binding tyrosyl residue (Y757) of gp130 required lower levels of gp130 stimulation for the maintenance of pluripotency compared to ES cells bearing a WT chimeric receptor (Burdon, T. et al. 1999 Dev Biol 210:30-43). However, subsequent studies have demonstrated that SOCS-3 (suppressor of cytokine signaling-3) also binds to Y757 of gp130, bringing into question the function of Shp-2 in this self-renewal model (Nicholson, S. E. et al. 2000 PNAS USA 97:6493-6498; Schmitz, J. et al. 2000 J Biol Chem 275:12848-12856). Thus, the role of Shp-2 in ES cell self-renewal and apoptosis remains unclear.

### SUMMARY OF THE INVENTION

This invention provides in one aspect a composition for inhibiting cell differentiation and apoptosis and enhancing self-renewal of a stem cell comprising a culture medium supplemented with at least one Shp-2 inhibitor.

One embodiment of the invention is also a method for the ex vivo expansion of stem cells comprising the steps of (a) providing stem cells isolated from a mammal, (b) contacting the stem cells with a culture medium comprising an Shp-2 inhibitor, and (c) incubating said stem cells in the presence of the Shp-2 inhibitor. Proliferation and perpetuation of the stem cell progeny can be carried out either in suspension cultures, or by allowing cells to adhere to a fixed substrate. Expansion can be done before or after transplantation, for example (1) expansion in vitro, then transplantation, (2) expansion in vitro, transplantation, then further expansion in vivo. The expansion of the stem cells can be in vivo, which can be done directly in the host without the need for transplantation.

Furthermore, another embodiment is a method for treating diseases comprising administering to a mammal stem cell progeny which have been treated with an Shp-2 inhibitor in order to expand their population. The treatment inhibits stem cell differentiation and apoptosis, thus resulting in increased self-renewal.

### BRIEF DESCRIPTION OF THE DRAWINGS

FIGS. 1A and 1B show that reconstitution with Shp-2 restores leukemia inhibitory factor (LIF)-stimulated P-Erk (extracellular signal regulated kinase). Each ES cell line was either unstimulated or stimulated with LIF 1000 U/mL for 20 minutes followed by immunoblot analysis for phospho-Erk (P-Erk) and Erk. The graph (FIG. 1B) is a densitometric representation of the fold-increase of P-Erk illustrated in the gel (FIG. 1A). Each value was obtained dividing the normalized, stimulated P-Erk value by the normalized, unstimulated P-Erk value.

FIGS. 2A and 2B are bar graphs that show that reconstitution with Shp-2 rescues ES cell differentiation. FIG. 2A. ES cell colonies were scored following 48 hours of culture without LIF. The number of differentiated colonies was divided by the total number of colonies to yield % differentiated colonies +/−S.E.M. FIG. 2B. Embryoid bodies (EBs) were scored for the presence or absence of hemoglobinized cells at day 8 to 10 of differentiation. The number of hemoglobinized EBs was divided by the total number of EBs to yield % hemoglobinization +/−S.E.M.

FIGS. 3A-3D are bar graphs that illustrate that reconstitution with Shp-2 rescues primitive and definitive hematopoiesis. EBs grown in primary differentiation culture were harvested, dissociated, and plated into secondary culture for: FIG. 3A. primitive erythroid (EryP) progenitors; FIG. 3B. definitive erythroid (EryD) progenitors; FIG. 3C. mixed progenitors; FIG. 3D. granulocyte/macrophage (GM) progenitors. Error bars represent S.E.M.

FIGS. 4A and 4B shows that LIF-stimulated Stat3 (signal transducer and activator of transcription 3) activity is greater in Shp-2^{-/-} cells. Each ES cell line was either unstimulated or stimulated with LIF 1000 U/mL for 5 or 10 minutes followed by immunoblot analysis for P-Stat3 and Stat3. The graph (FIG. 4B) is a representation of densitometric values of P-Stat3 band intensities (FIG. 4A) normalized to Stat3 band intensities.

FIG. 5 is a bar graph illustrating that Shp-2 expression is inversely proportional to 2° EB formation. EBs grown for 7 days in primary differentiation culture were harvested, dissociated, and plated into secondary culture for 2° EBs. 2° EBs were scored on day 7 of secondary culture. Error bars represent S.E.M.

FIGS. 6A and 6B shows that Shp-2 expression increases ES cell apoptosis. ES cells were cultured on gelatinized plates for 96 hours without change or supplementation of media followed by trypsinization, staining with annexin V-FITC and propidium iodide (PI), and FACS analysis. FIG. 6A. FACS analysis results from representative experiment. FIG. 6B. Graphic representation of four independent experiments. Values for % annexin V positive cells were calculated by adding the values of the upper right quadrant (annexin V+/PI+) and lower right quadrant (annexin V+/PI−). *p≦0.05 when comparing WT to Shp-2^{-/-} or Shp-2⁰.

FIGS. 7A and 7B are schematic diagrams of aberrant ES cell function in the absence of functional Shp-2 (FIG. 7A) and correction upon reintroduction of WT Shp-2 (FIG. 7B).

### DETAILED DESCRIPTION OF THE PREFERRED EMBODIMENT

In one aspect, the present invention includes agonists and antagonists of Shp-2.

The present invention further includes assays for identifying an antagonist or an agonist of a Shp-2.

In another embodiment, the invention includes an assay for identifying an antagonist or agonist of a Shp-2 by cultivating a Shp-2-expressing stem cell line or progenitor cell line in the presence of a candidate antagonist or agonist, and monitoring the differentiation, self-renewal, and apoptosis of the progenitor cells.

The invention further includes a method for inhibiting differentiation and apoptosis and stimulating self-renewal of undifferentiated stem cells, by contacting the undifferentiated stem cells with an antagonist of Shp-2.

In an additional aspect, the invention includes a method for the induction of stem cell differentiation, by contacting the stem cells with an agonist of Shp-2.

In another aspect, the invention includes a method for expansion of undifferentiated stem cells in cell culture, by cultivating stem cells in the presence of an antagonist of Shp-2.

In yet another aspect, the invention includes a method for the expansion of undifferentiated stem cells in vivo by administering to a patient an antagonist of Shp-2, and a stem cell growth factor.

In yet another aspect, the invention includes a method of treatment of a disease, by administering stem cell progeny to a mammal, wherein the progeny have been treated with an Shp-2 inhibitor to expand their population by inhibiting stem cell differentiation and apoptosis and stimulating their self-renewal.

In a preferred method, the stem cells are selected from: pluripotent stem cells, such as embryonic stem cells or embryonic germ cells; and lineage restricted stem cells such as, but not limited to: hematopoietic stem cells, muscle stem cells, nerve stem cells, liver stem cells, or skin dermal sheath stem cells.

Overview

Shp-2 is a widely expressed non-transmembrane protein tyrosine phosphatase. Previous studies demonstrated that homozygous mutant (Shp-2^{-/-}) embryonic stem (ES) cells bearing a targeted exon 3 deletion exhibit decreased hematopoiesis and increased sensitivity to leukemia inhibitory factor (LIF). However, the mechanism of Shp-2 action in hematopoiesis and LIF signaling within ES cells was unclear. To characterize the role of Shp-2 in stem cell activities, we transfected Shp-2^{-/-} ES cells with a wild-type Shp-2 cDNA expression construct and selected three clones with varying levels of Shp-2 expression for evaluation in functional and biochemical assays. Reintroduction of Shp-2 rescued LIF-stimulated Erk (extracellular signal regulated kinase) activation, ES cell differentiation, and hematopoiesis as assayed by immunoblotting, ES cell colony differentiation in vitro, and hemoglobinization of 1° embryoid bodies (EBs), respectively. Rescue of primitive erythropoiesis and definitive hematopoiesis was also observed in secondary plating assays and in expression analysis of hematopoietic cell-specific genes. Furthermore, we detected higher LIF-stimulated phospho-Stat3 (signal transducer and activator of transcription 3) levels in Shp-2^{-/-} ES cells compared to that in Shp-2^{-/-} ES cells expressing WT Shp-2. Functionally, LIF-stimulated phospho-Stat3 levels were proportional to ES cell self-renewal and survival. Collectively, these experiments unequivocally define a critical role of Shp-2 in hematopoiesis and in the ES cell functions of differentiation, self-renewal, and programmed cell death. Mechanistically, Shp-2 appears to operate by inhibiting signaling events that maintain ES cells in an undifferentiated, self-renewing state, such as the LIF-stimulated Jak-Stat3 pathway.

Screening Assays for Compounds that Modulate Shp-2 Expression or Activity

The following assays identify compounds that interact with Shp-2. Also described are assays that identify compounds that interfere with the interaction of Shp-2 with its natural ligands, transmembrane or intracellular proteins involved in Shp-2-mediated signal transduction, and to compounds which modulate the activity of Shp-2 gene (see, for example, Sui, G. et al. 2002 PNAS USA 99:5515-5520). Assays may additionally be utilized which identify compounds which bind to Shp-2 gene regulatory sequences and which may modulate Shp-2 gene expression (see, for example, Platt, K. A. 1994 J Biol Chem 269:28558-28562).

The compounds which may be screened include, but are not limited to, peptides, antibodies and fragments thereof, and other organic compounds (such as for example, peptidomimetics) that bind to the Shp-2 and inhibit the activity triggered by the natural ligand (i.e., antagonists); as well as peptides, antibodies or fragments thereof, and other organic compounds that mimic the active site of the Shp-2 (or a portion thereof) and bind to and “neutralize” a natural ligand.

Such compounds may include, but are not limited to, peptides such as, for example, soluble peptides, including but not limited to members of random peptide libraries (see, for example, Lam, K. S. et al. 1991 Nature 354:82-84; Houghten, R. et al. 1991 Nature 354:84-86), and combinatorial chemistry-derived molecular library made of D- and/or L-configuration amino acids, phosphopeptides and antibodies. In one embodiment, the antibodies include polyclonal, monoclonal, humanized, anti-idiotypic, chimeric or single chain antibodies. Moreover, FAb, F(ab′)₂ and FAb expression library fragments, and epitope-binding fragments thereof are also contemplated. Other embodiments include small organic or inorganic molecules which may be screened, as described herein.

Other compounds which can be screened in accordance with the invention include, but are not limited to, small organic molecules and polynucleotides that are able to gain entry into an appropriate cell and affect the expression of the Shp-2 gene or some other gene involved in the Shp-2 signal transduction pathway. Compounds that affect the activity of the Shp-2 by inhibiting the enzymatic activity of the Shp-2 or the activity of some other intracellular factor involved in the Shp-2 signal transduction pathway are also within the scope of the invention. Compounds that affect the activity of the Shp-2 by enhancing the enzymatic activity of the Shp-2 or the activity of some other intracellular factor involved in the Shp-2 signal transduction pathway are within the scope of the invention as well.

Computer modeling and searching technologies permit identification of compounds, or the improvement of already identified compounds, that can modulate Shp-2 expression or activity. Having identified such a compound or composition, the active sites or regions can be identified. Such active sites might typically be ligand-binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the Shp-2 polypeptide the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds of Shp-2 can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential Shp-2 modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of Shp-2 natural ligands, Shp-2, and related transduction and transcription factors will be apparent to those of skill in the art.

Examples of molecular modeling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen, et al. 1988 Acta Pharmaceutical Fennica 97:159-166; Ripka, 1988 New Scientist 54-57; McKinaly and Rossmann 1989 Annu Rev Pharmacol Toxicol 29:111-122; Perry and Davies 1989 OSAR: Quantitative Structure-Activity Relationships in Drug Design pp. 189-193 Alan R. Liss, Inc.; Lewis and Dean 1989 Proc R Soc Lond 236:125-140 and 141-162; and, with respect to a model receptor for nucleic acid components, Askew, et al. 1989 J Am Chem Soc 111:1082-1090. Other computer programs that screen and graphically depict chemicals are available from companies such as BioDesign, Inc. (Pasadena, Calif), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators, preferably inhibitors.

Compounds identified via assays such as those described herein may be useful, for example, in modulating stem cell self-renewal, survival and differentiation.

In Vitro Cell-Free Screening Assays for Compounds that Bind to Shp-2.

In vitro systems may be designed to identify compounds capable of interacting with Shp-2. These compounds may be useful, for example, in modulating the activity of wild-type and/or mutant Shp-2 gene products. In addition, these compounds may be useful in elaborating the biological function of the Shp-2 or may be utilized in screens for identifying compounds that disrupt normal Shp-2 interactions. Alternatively, the compounds themselves may disrupt such interactions.

The assays used to identify compounds that bind to Shp-2 involve preparing a reaction mixture of Shp-2 and the test compound under conditions and for a time sufficient to allow the two components to interact, thus forming a complex which can be removed and/or detected in the reaction mixture. The Shp-2 species used can vary depending upon the goal of the screening assay. For example, where antagonists of the natural ligand are sought, the full length Shp-2, or a peptide corresponding to the Shp-2 active site, or a fusion protein containing the Shp-2 active site fused to a protein or polypeptide that affords advantages in the assay system can be utilized. Such assay system may be, but not limited to labeling, isolation of the resulting complex, etc.

The screening assays can be conducted in a variety of ways. For example, one method to conduct such an assay would involve anchoring the Shp-2 protein, polypeptide, peptide or fusion protein or the test substance onto a solid phase and detecting Shp-2/test compound complexes anchored on the solid phase at the end of the reaction. In one embodiment of such a method, the Shp-2 reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface. In one embodiment, a labeled antibody specific for the previously nonimmobilized component is used. The antibody, in turn, may be directly labeled or indirectly labeled with a labeled anti-Ig antibody.

Alternatively, a reaction can be conducted in a liquid phase, the reaction products separated from unreacted components, and complexes detected. In one embodiment, an immobilized antibody specific for the Shp-2 protein, polypeptide, peptide or fusion protein or the test compound is used to anchor any complexes formed in solution, and a labeled antibody specific for the other component of the possible complex is used to detect anchored complexes.

Alternatively, cell-based assays, membrane vesicle-based assays and membrane fraction-based assays can be used to identify compounds that interact with Shp-2. To this end, cell lines that express Shp-2, or cell lines that have been genetically engineered to express Shp-2 can be used.

Assays for Intracellular Proteins that Interact with the Shp-2.

Any method suitable for detecting protein-protein interactions may be employed for identifying transmembrane proteins or intracellular proteins that interact with Shp-2. Among the traditional methods which may be employed are co-immunoprecipitation, crosslinking and co-purification through gradients or chromatographic columns of cell lysates or proteins obtained from cell lysates and the Shp-2 to identify proteins in the lysate that interact with the Shp-2. For these assays, the Shp-2 component used can be a full-length Shp-2, a peptide corresponding to the active site of Shp-2, or a fusion protein containing the active site of Shp-2.

Once isolated, such an intracellular protein can be identified and can, in turn, be used, in conjunction with standard techniques, to identify proteins with which it interacts. For example, at least a portion of the amino acid sequence of an intracellular protein which interacts with the Shp-2 can be ascertained using techniques well known to those of skill in the art, such as via the Edman degradation technique (see, for example, Creighton, 1983 Proteins: Structures and Molecular Principles, W. H. Freeman & Co. N.Y. pp. 34-49). The amino acid sequence obtained may be used as a guide for generating oligonucleotide mixtures that can be used to screen for gene sequences encoding such intracellular proteins. Screening may be accomplished, for example, by standard hybridization or well-known PCR techniques. Techniques for the generation of oligonucleotide mixtures and the screening are well-known (see, for example, Ausubel, F. M. et al. eds. 1989 Current Protocols in Molecular Biology Green Publishing Associates Inc., and John Wiley & sons, Inc. New York; and Innis, M. et al., eds. 1990 PCR Protocols: A Guide to Methods and Applications, Academic Press, Inc., New York).

Additionally, methods may be employed which result in the simultaneous identification of genes which encode the transmembrane or intracellular proteins interacting with Shp-2. These methods include, for example, probing expression libraries, in a manner similar to the well-known technique of antibody probing of λgt11 libraries, using labeled Shp-2 protein, or a Shp-2 polypeptide, peptide or fusion protein. Such fusion protein may be a Shp-2 polypeptide or Shp-2 domain fused to a marker such as an enzyme, fluor, luminescent protein, or dye. Alternatively, such fusion protein may be a Shp-2 polypeptide or Shp-2 domain fused to an Ig-Fc domain.

One method which detects protein interactions in vivo, the two-hybrid system, is described in detail for illustration only and not by way of limitation. Several versions of this system have been described (Chien et al. 1991 PNAS USA 88:9578-9582; Yamada, M. et al. 2001 J Biochem (Tokyo) 130:157-65), and it is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to a Shp-2 nucleotide sequence encoding Shp-2, a Shp-2 polypeptide, peptide or fusion protein, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene, such as, for example, HBS or lacZ whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, Shp-2 may be used as the bait gene product. Total genomic or cDNA sequences are fused to the DNA encoding an activation domain. This library and a plasmid encoding a hybrid of a bait Shp-2 gene product fused to the DNA-binding domain are cotransformed into a yeast reporter strain, and the resulting transformants are screened for those that express the reporter gene. For example, and not by way of limitation, a bait Shp-2 gene sequence, such as the open reading frame of Shp-2 (or a domain of Shp-2), can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait Shp-2 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait Shp-2 gene-GAL4 fusion plasmid into a yeast strain which contains a lacZ gene driven by a promoter which contains GAL4 activation sequence. A cDNA encoded protein, fused to GAL4 transcriptional activation domain, that interacts with bait Shp-2 gene product will reconstitute an active GAL4 protein and thereby drive expression of the HIS3 gene. Colonies which express HIS3 can be detected by their growth on Petri dishes containing semi-solid agar based media lacking histidine. The cDNA can then be purified from these strains, and used to produce and isolate the bait Shp-2 gene-interacting protein using techniques routinely practiced in the art.

Assays for Compounds that Interfere with Shp-2/Intracellular or Shp-2/Transmembrane Macromolecule Interaction.

The macromolecules that interact with the Shp-2 are referred to, for purposes of this discussion, as “binding partners”. These binding partners are likely to be involved in the Shp-2 signal transduction pathway, and therefore, in the role of Shp-2 in modulation of stem cell self-renewal, survival and differentiation. Therefore, it is desirable to identify compounds that interfere with or disrupt the interaction of such binding partners with Shp-2 which may be useful in regulating the activity of the Shp-2 and control stem cell self-renewal, survival and differentiation associated with Shp-2 activity.

The basic principle of the assay systems used to identify compounds that interfere with the interaction between the Shp-2 and its binding partner or partners involves preparing a reaction mixture containing Shp-2 protein, polypeptide, peptide or fusion protein as described above, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture. Alternatively, the test compound may be added at a time subsequent to the addition of the Shp-2 moiety and its binding partner. Control reaction mixtures are incubated without the test compound or with a placebo. The formation of any complexes between the Shp-2 moiety and the binding partner is then detected. The formation of a complex in the control reaction, but not in the reaction mixture containing the test compound, indicates that the compound interferes with the interaction of the Shp-2 and the interactive binding partner. Additionally, complex formation within reaction mixtures containing the test compound and normal Shp-2 protein may also be compared to complex formation within reaction mixtures containing the test compound and a mutant Shp-2. This comparison may be important in those cases wherein it is desirable to identify compounds that disrupt interactions of mutant but not normal Shp-2.

The assay for compounds that interfere with the interaction of the Shp-2 and binding partners can be conducted in a heterogeneous or homogeneous format. Heterogeneous assays involve anchoring either the Shp-2 moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance. In one embodiment, the test substance is added to the reaction mixture prior to the Shp-2 moiety and interactive binding partner. In another embodiment, the test substance is added to the reaction mixture simultaneously with the Shp-2 moiety and interactive binding partner. Alternatively, test compounds that disrupt preformed complexes, can be tested by adding the test compound to the reaction mixture after complexes have been formed. The various formats are described briefly below.

In a heterogeneous assay system, either the Shp-2 moiety or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the Shp-2 gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed, for example, by washing and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface. In one embodiment, a labeled antibody specific for the initially non-immobilized species may be used. The antibody, in turn, may be directly labeled or indirectly labeled with a labeled anti-Ig antibody. Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected. Using an immobilized antibody specific for one of the binding components to anchor any complexes formed in solution, and a labeled antibody specific for the other partner to detect anchored complexes is contemplated. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the Shp-2 moiety and the interactive binding partner is prepared in which either the Shp-2 or its binding partners is labeled, but the signal generated by the label is quenched due to formation of the complex (see, for example, U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt Shp-2/intracellular binding partner interaction can be identified.

In a particular embodiment, a Shp-2 fusion can be prepared for immobilization. For example, the Shp-2 or a peptide fragment, for example, corresponding to the Shp-2 active site, can be fused to a glutathione-S-transferase (GST) gene using a fusion vector, such as pGEX-5×-1, in such a manner that its binding activity is maintained in the resulting fusion protein. The interactive binding partner can be purified and used to raise a monoclonal antibody, using methods routinely practiced in the art. This antibody can be labeled with a radioactive isotope, for example ¹²⁵I, by methods routinely practiced in the art. In a heterogeneous assay, the GST-Shp-2 fusion protein may be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the Shp-2 gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-Shp-2 fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the Shp-2/binding partner interaction can be detected by adding the labeled antibody and measuring the radioactivity associated with the beads.

In another embodiment of the invention, these same techniques can be employed using peptide fragments that correspond to the binding domains of the Shp-2 and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

For example, and not by way of limitation, a Shp-2 gene product can be anchored to a solid material as described, above, by making a GST-Shp-2 fusion protein and allowing it to bind to glutathione agarose beads. The interactive binding partner can be labeled with a radioactive isotope, such as 35S, and cleaved with a proteolytic enzyme such as trypsin. Cleavage products can then be added to the anchored GST-Shp-2 fusion protein and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative proteins using recombinant DNA technology.

Cell- and Membrane-Based Screening Assays for Shp-2 Inhibitors

Compounds, including but not limited to binding compounds identified via assay techniques such as those described in the preceding sections above can be tested for the ability to modulate stem cell self-renewal, survival and differentiation. The assays described above can identify compounds which affect Shp-2 activity. Compounds that bind to Shp-2, inhibit binding of the natural ligand, and either activate signal transduction (agonists) or block activation (antagonists) are within the scope of the present invention. Compounds that bind to a natural ligand of Shp-2 and neutralize ligand activity are also within the scope of the present invention. Compounds that affect Shp-2 gene activity are also contemplated. Such compounds may be proteins or small organic molecules. However, it should be noted that the assays described can also identify compounds that modulate Shp-2 signal transduction such as upstream or downstream signaling events. The identification and use of such compounds which affect another step in the Shp-2 signal transduction pathway in which the Shp-2 gene product is involved and, by affecting this same pathway may modulate the effect of Shp-2 on the modulation of stem cell self-renewal, survival and differentiation are within the scope of the invention. Such compounds can be used as part of a method for the modulation of stem cell self-renewal, survival and differentiation.

Cell-based systems, membrane vesicle-based systems, and membrane fraction-based systems can be used to identify compounds which may act to modulate stem cell self-renewal, survival and differentiation. Such systems can include, for example, recombinant or non-recombinant cells, such as cell lines, which express the Shp-2 gene. In addition, expression host cells genetically engineered to express a functional Shp-2 and to respond to activation by a natural Shp-2 ligand can be used as an end point in the assay. Such activation can be measured by a chemical or phenotypic change, induction of another host cell gene, change in ion flux, phosphorylation of host cell proteins, etc.

In utilizing such cell-based systems, cells may be exposed to a compound suspected of exhibiting an ability to modulate stem cell self-renewal, survival and differentiation, at a sufficient concentration and for a time sufficient to elicit chemical or phenotypic change, induction of another host cell gene, change in ion flux, phosphorylation of host cell proteins, etc. in the exposed cells. After exposure, the cells can be assayed to measure alterations in the expression of the Shp-2 gene. For example, cell lysates may be assayed for Shp-2 mRNA transcripts or for Shp-2 protein expressed in the cell. Compounds which regulate or modulate expression of the Shp-2 gene are good candidates as modulators of stem cell self-renewal, survival and differentiation. Alternatively, the cells are examined to determine whether cellular phenotypes has been altered to resemble a more differentiated type, or if the level of cellular apoptosis has been altered, or if cell self-renewal has been altered. Still further, the expression and/or activity of components of the signal transduction pathway of which Shp-2 is a part, or the activity of the Shp-2 signal transduction pathway itself can be assayed.

For example, after exposure, the cell lysates can be assayed for the presence of phosphorylation of host cell proteins, as compared to lysates derived from unexposed control cells. The ability of a test compound to inhibit phosphorylation of host cell proteins in these assay systems indicates that the test compound inhibits signal transduction initiated by Shp-2 activation. The cell lysates can be readily assayed using a Western blot format well known in the art (see, for example, Glenney et al. 1988 J Immunol Methods 109:277-285; Frackelton et al. 1983 Mol Cell Biol 3:1343-1352). Alternatively, an ELISA format could be used in which a particular host cell protein involved in the Shp-2 signal transduction pathway is immobilized using an anchoring antibody specific for the target host cell protein, and the presence or absence of a phosphorylated peptide residue on the immobilized host cell protein is detected using a labeled antibody (see, King et al. 1993 Life Sciences 53:1465-1472). In yet another approach, ion flux, such as calcium, potassium, sodium, bicarbonate, chloride ion flux, can be measured as an end point for Shp-2 stimulated signal transduction.

In general, other cell-based screening procedures of the invention involve providing appropriate cells which express a Shp-2 polypeptide. Such cells include cells from mammals, yeast, Drosophila or E. coli. In particular, a polynucleotide encoding the Shp-2 is employed to transfect cells to thereby express a Shp-2. The expressed Shp-2 is then contacted with a test compound to observe binding, stimulation or inhibition of a functional response.

One such screening procedure involves the use of melanophores which are transfected to express a Shp-2 polypeptide. Such a screening technique is described in PCT WO 92/01810, published Feb. 6, 1992. Such an assay may be employed to screen for a compound which inhibits activation of Shp-2 by contacting the melanophore cells which encode the Shp-2 polypeptide with both an Shp-2 ligand, and a compound to be screened. Inhibition of the signal generated by the ligand indicates that a compound is a potential antagonist for the Shp-2, as it inhibits activation of the Shp-2 polypeptide.

The technique may also be employed for screening of compounds which activate the Shp-2 by contacting such cells with compounds to be screened and determining whether such compound generates a signal, as it activates the Shp-2 polypeptide.

Other screening techniques include the use of cells which express a Shp-2 in a system which measures extracellular pH changes caused by Shp-2 activation. In this technique, compounds may be contacted with cells expressing an Shp-2 polypeptide. A second messenger response, for example, signal transduction or pH changes, is then measured to determine whether the potential compound activates or inhibits the Shp-2 polypeptide.

Another method involves screening for compounds which are antagonists, and thus inhibit activation of a Shp-2 polypeptide by determining inhibition of binding of a labeled Shp-2 ligand, in the cells which express Shp-2. Such a method involves transfecting a eukaryotic cell with a DNA encoding an Shp-2 polypeptide such that the cell expresses the Shp-2 polypeptide. Alternatively a eukaryotic cell that expresses the Shp-2 may be used. The cell is then contacted with a potential antagonist in the presence of a labeled form of an Shp-2 ligand. The amount of labeled ligand bound to the Shp-2 is measured. If the compound binds to the Shp-2, the binding of labeled ligand to the Shp-2 is inhibited as determined by a reduction of labeled ligand which binds to the Shp-2. This method is called a binding assay.

Another such screening procedure involves the use of eukaryotic cells which are transfected to express Shp-2 (or use of eukaryotic cells that express the Shp-2). The cells are loaded with an indicator dye that produces a fluorescent signal when bound to calcium, and the cells are contacted with a test substance and a Shp-2 agonist. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist (or agonist) for the Shp-2 polypeptide.

Another such screening procedure involves use of eukaryotic cells which are transfected to express the Shp-2 of the present invention (or use of eukaryotic cells that express the Shp-2), and which are also transfected with a reporter gene construct that is coupled to activation of the Shp-2 polypeptide behind an appropriate promoter. Such reporter gene may be for example, luciferase or beta-galactosidase. The cells are contacted with a test substance and an Shp-2 agonist and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the Shp-2 polypeptide.

Another such screening technique for antagonists or agonists involves introducing RNA encoding an Shp-2 polypeptide into Xenopus oocytes to transiently or stably express the Shp-2 polypeptide. The oocytes are then contacted with an Shp-2 ligand and a compound to be screened. Inhibition or activation of the Shp-2 is then determined by detection of a signal, such as, cAMP, calcium, proton, or other ions.

Another method involves screening for Shp-2 polypeptide inhibitors by determining inhibition or stimulation of Shp-2 polypeptide-mediated cAMP and/or adenylate cyclase accumulation or diminution. Such a method involves transiently or stably transfecting an eukaryotic cell with an Shp-2 polynucleotide to express the Shp-2 or using a eukaryotic cell that expresses the Shp-2. The cell is then exposed to potential antagonists in the presence of Shp-2 polypeptide ligand. The amount of cAMP accumulation is then measured, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist binds the Shp-2 polypeptide, and thus inhibits Shp-2 polypeptide activity, the levels of Shp-2 polypeptide-mediated cAMP, or adenylate cyclase activity, will be reduced or increased.

The present invention also provides a method for determining whether a ligand not known to be capable of binding to Shp-2 polypeptide can bind to such phosphatase. Such method comprises contacting a eukaryotic cell which expresses an Shp-2 polypeptide with the ligand, under conditions permitting binding of candidate ligands to Shp-2, and detecting the presence of a candidate ligand bound to the Shp-2. The systems hereinabove described for determining agonists and/or antagonists may also be employed for determining ligands which bind to the Shp-2.

Potential Shp-2 Antagonists

Examples of potential Shp-2 polypeptide antagonists include antibodies or, in some cases, oligonucleotides, which bind to the Shp-2 but do not elicit a second messenger response such that the activity of the Shp-2 polypeptide is prevented.

Potential antagonists also include proteins which are closely related to a ligand of the Shp-2 polypeptide. For example, a fragment of the ligand, which have lost biological function and when binding to the Shp-2 polypeptide, elicit no response is within the scope of the present invention.

A potential antagonist also includes an antisense construct prepared through the use of antisense technology (see, for example., WO 01/07655). Antisense technology can be used to control gene expression through triple-helix formation or antisense DNA or RNA, both methods of which are based on binding of a polynucleotide to DNA or RNA. For example, the 5′ coding portion of the polynucleotide sequence, which encodes for the Shp-2 polypeptide, is used to design an antisense RNA oligonucleotide of from about 10 to 40 base pairs in length. A DNA oligonucleotide is designed to be complementary to a region of the gene involved in transcription (triple helix—see Lee, et al. 1979 Nucl Acids Res 6:3073; Cooney, et al. 1988 Science 241:456; and Dervan, et al. 1991 Science 251:1360), thereby preventing transcription and production of a Shp-2 polypeptide. The antisense RNA oligonucleotide hybridizes to the mRNA in vivo and blocks translation of the mRNA molecule to a Shp-2 polypeptide (antisense—Okano, J. 1991 Neurochem 56:560; Oligonucleotides as antisense inhibitors of gene expression, 1988, CRC Press, Boca Raton, Fla.). The oligonucleotides described above can also be delivered to cells such that the antisense RNA or DNA may be expressed in vivo to inhibit production of a Shp-2 polypeptide. See, for example, U.S. Pat. No. 6,200,807.

Another potential antagonist is a double-stranded RNA that triggers silencing of the Shp-2 gene expression by RNA-mediated interference (RNAi) through the destruction of mRNA complementary to the sequence comprising the RNAi molecule. Such RNAi molecule may be derived from exonic or coding sequence of the Shp-2 gene (see, for example, Sui, G. et al. 2002 PNAS USA 99:5515-5520; and WO 02/16620).

Another potential antagonist is a small molecule which binds to an Shp-2 polypeptide, making it inaccessible to ligands such that normal biological activity is prevented. Examples of small molecules include, but are not limited to, small peptides or peptide-like molecules.

Therapeutic Uses of Shp-2 Antagonists

Shp-2 antagonists identified using methods described herein can be used in culture for ex vivo expansion of stem cells. The cells for culturing can be obtained from a variety of tissues, including but not limited to blood, bone marrow, muscle tissue, nerve tissue, liver, skin, etc. The ex vivo expanded stem cells of present invention can be used for treatment of mammals (humans or animals) anticipating or having undergone exposure to chemotherapeutic agents, other agents which damage cycling stem cells, or radiation exposure. Shp-2 antagonists described herein can be used for the improvement of the stem cell maintenance or expansion cultures for auto and allo-transplantation procedures or for gene transfer.

In one embodiment of the present invention cells for culturing are obtained from a patient in need of a treatment. In another embodiment the cells for culturing are of embryonic origin. The cells in culture are then treated with an Shp-2 inhibitor to inhibit their differentiation, and to increase their self-renewal and survival. Preferably, such inhibition is reversible. After ex vivo expanding using an Shp-2 inhibitor of the present invention, the stem cell culture is transplanted into a patient to alleviate the symptoms of a disease, tissue/organ degeneration or trauma. In one aspect, this invention also relates to a method of generating cells for autologous transplantation. In another aspect, the stem cell treated with an Shp-2 inhibitor of the present invention may be used for the purposes of drug screening of putative therapeutic agents targeted at different systems. In another aspect, an Shp-2 inhibitor of the present invention may be introduced into a patient in need of in vivo expansion of stem cells, preferably in the presence of a stem cell growth factor.

In one embodiment, the stem cells obtained and expanded as described herein are hematopoictic stem cells. Bone marrow transplantation and circulating blood stem cell transplantation (hereafter both referred as hematopoietic stem cell transplantation, HSCT) are the treatment of choice in several disorders, including malignancies, Severe Combined Immune Deficiencies (SCID), congenitally or genetically determined hematopoietic abnormalities, anemia, aplastic anemia, leukemia and osteoporosis.

Autologous HSCT defines a stem cell transplantation in which donor and recipient are the same individual. Non-autologous HSCT comprises HSCT in which donor and recipient are different individuals, either genetically identical (syngenic) or genetically different (allogenic).

Non-autologous HSCT is subject to immunological reactions, such as graft-versus-host disease and host-versus-graft reaction (graft rejection). The mechanisms of graft rejections are not completely known, but in addition to immune mechanisms, hematopoietic stem cells may also be rejected by natural killer (NK) cells. The recipient's immune system must be ablated to permit successful non-autologous HSCT.

To prepare for HSCT, the recipient's immune system is destroyed with radiation and/or chemotherapy. This procedure not only prevents non-autologous graft rejection but also serves to kill leukemic or other malignant cells if that is the patient's disease. Following HSCT, hematopoietic and immune cells of the recipients are replaced with those expanded ex vivo in the presence of an identified Shp-2 inhibitor of the present invention.

In another embodiment, the stem cells obtained and expanded as described herein are neural stem cells treated with an Shp-2 inhibitor of the present invention. Any suitable tissue source may be used to derive the neural stem cells of this invention. The role of stem cells is to replace cells that are lost by natural cell death, injury or disease. CNS disorders encompass numerous afflictions including neurodegenerative diseases such as Alzheimer's Disease, Multiple Sclerosis (MS), Huntington's Disease, Amyotrophic Lateral Sclerosis, and Parkinson's Disease. CNS disorders also encompass acute brain injury such as stroke, head injury, cerebral palsy. A large number of CNS dysfunctions such as depression, epilepsy, schizophrenia, neurosis and psychosis may also be treated using stem cells expanded as described herein.

In another embodiment, the stem cells obtained and expanded as described herein are liver stem cells which can be used to treat degenerative liver diseases or inherited deficiencies of liver function, and for artificial livers, gene therapy, drug testing and vaccine production.

### Embryonic Stem Cell Differentiation and Hematopoiesis

We transfected Shp-2^{-/-} ES cells with the WT Shp-2 cDNA and generated cell lines expressing various levels of the WT Shp-2 protein. Using these cell lines, the defective differentiation capacity and leukemia inhibitory factor (LIF) hypersensitivity observed using the targeted Shp-2^{-/-} ES cells was confirmed to be due to lack of functional Shp-2. To define the mechanism for the observed defect in differentiation and LIF hypersensitivity, we found that the Shp-2′ ES cells have increased LIF-stimulated phospho-Stat3 (signal transducer and activator of transcription 3) levels. Functionally, the Shp-2^{-/-} cells had dramatically increased levels of self-renewal as assayed by 2° embryoid bodies (EBs) formation and increased survival as assayed by annexin V binding. Taken together, these results clearly demonstrate that Shp-2 is necessary and sufficient for the differentiation of ES cells to hematopoietic cells. Furthermore, we discovered that biochemical downregulation of LIF-stimulated phospho-Stat3 activity by Shp-2 is functionally correlated with increased ES cell self-renewal and survival.

Generation of Rescue Cell Lines

Analysis of Shp-2^{-/-} ES cells has revealed several interesting findings including decreased differentiation and hematopoiesis. To verify that the observed phenotypes were due to the lack of functional Shp-2 rather than due to a neomorphic effect of the residual mutant protein, rescue analysis was performed. The Shp-2^{-/-} ES cell line, IC3, was transfected with the plasmid phβA-Shp-2 and subjected to selection in hygromycin. Three clones demonstrating various levels of Shp-2 expression (high, low, and none) were used for further analysis. To compare the levels of Shp-2 protein expression, clarified total protein extract from each cell line was immunoprecipitated and blotted with anti-Shp-2 antibody. The expression levels of WT (64 kDa) Shp-2 in the selected clones: Shp-2^{Hi}, Shp-2^{Lo}, and Shp-2⁰ (a transfected and selected clone which failed to express wild-type Shp-2 and serves as a negative control) were compared to wild-type and IC3 (Shp-2^{-/-}) cells. As observed previously, the mutant (57 kDa) Shp-2 protein was expressed at a lower level compared to the WT protein. Additionally, although expression of the WT Shp-2 protein was achieved in the Shp-2^{Hi} and Shp-2^{Lo} cell lines, the amount of ectopic WT protein was less than that observed in the original WT cell line.

Rescue of Erk Activity

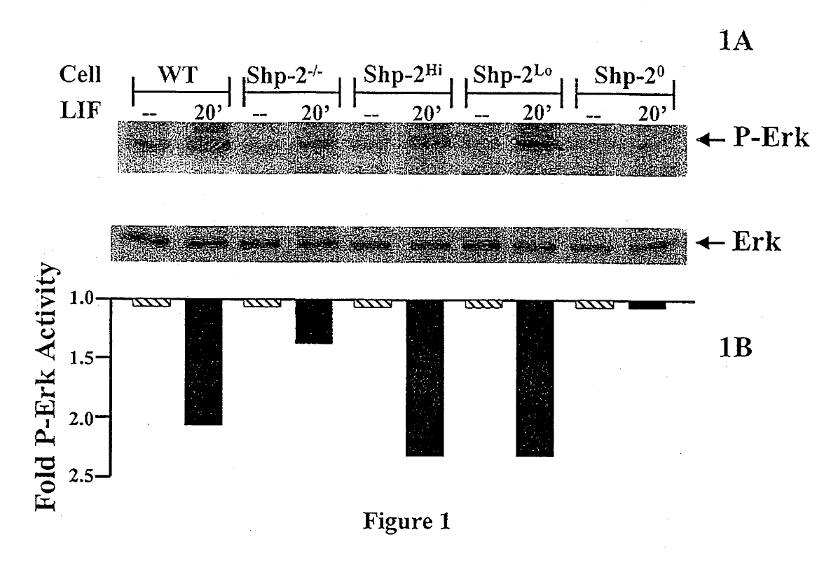

One of the demonstrated biochemical defects in Shp-2^{-/-} ES cells is decreased Erk activity in response to receptor stimulation (Qu, C. K. et al. 1997 Mol Cell Biol 17:5499-5507). We sought to determine if reconstitution with wild-type Shp-2 could rescue Erk activity in response to LIF stimulation. ES cell lines were “starved” in LIF-free, serum-free media for 6 hours (this amount of time was adequate for down-regulation of LIF-responsive effects without upregulation of endogenous LIF production). The cells were either unstimulated or stimulated with LIF (1000 U/ml) for 20 minutes. Protein extracts were analyzed for the amount of phospho-Erk as a measure of Erk activity. As shown in FIG. 1, phospho-Erk was increased in response to LIF stimulation in wild-type cells and the transfected cell lines expressing wild-type Shp-2, Shp-2^{Hi} and Shp-2^{Lo}. The negative control cell line, Shp-2⁰, exhibited minimal LIF-induced phospho-Erk, as observed with the IC3 (Shp-2^{-/-}) cells. The densitometric data representing band intensity are represented graphically. These results demonstrated that the transfected wild-type Shp-2 was functional biochemically in the Shp-2^{Hi} and Shp-2^{Lo} cells. Likewise, the Shp-2⁰ cell line behaved similarly to the parental Shp-2^{-/-} cell line and was used as the negative control for subsequent experiments.

Shp-2 Rescues ES Cell Differentiation

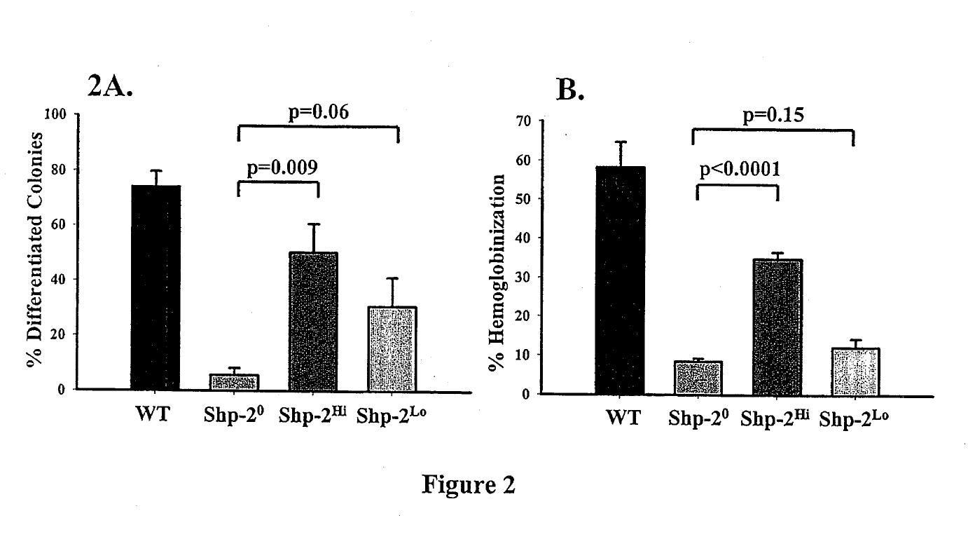

One measure of the differentiation capacity of ES cells is the development of flattened, fibroblast-like outgrowths from ES cell colonies upon the withdrawal of LIF. Each ES cell line was cultured on gelatinized tissue culture plates at colony dilution in LIF-containing medium for 6-8 days followed by growth in LIF-deficient media for an additional 48 hours. Wild-type ES cells consistently produced 60-70% of colonies with differentiated morphology in contrast to that of the negative control cell line, Shp-2⁰, which produced less than 5% differentiated colonies (FIG. 2A). Upon reconstitution with wild-type Shp-2, the defective differentiation phenotype was rescued (FIG. 2A). The lower level of differentiation for the Shp-2^{Lo} cell line is expected as this cell line has a lower level of Shp-2 expression. These results emphasized that reintroduction of WT Shp-2 is necessary and sufficient to support ES cell differentiation.

To evaluate further the specific differentiation of ES cells into hematopoietic cells, in vitro hematopoietic assays were performed. ES cells plated in semisolid culture grow into spheres of cells called 1° EBs. Differentiation into hematopoietic cells is particularly easy to detect as they become hemoglobinized and are red under light microscopy. As seen in FIG. 2B, 50-60% of the EBs developed from wild-type cells contained hemoglobinized cells compared to only 10% of the EBs developed from the negative control cell line, Shp-2⁰. This defective phenotype was corrected in the presence of wild-type Shp-2 in a dose-dependent manner as approximately 30% and 15% hemoglobinized EBs were observed using the Shp-2^{Hi} and Shp-2^{Lo} cell lines, respectively.

Shp-2 Rescues Primitive and Definitive Hematopoiesis

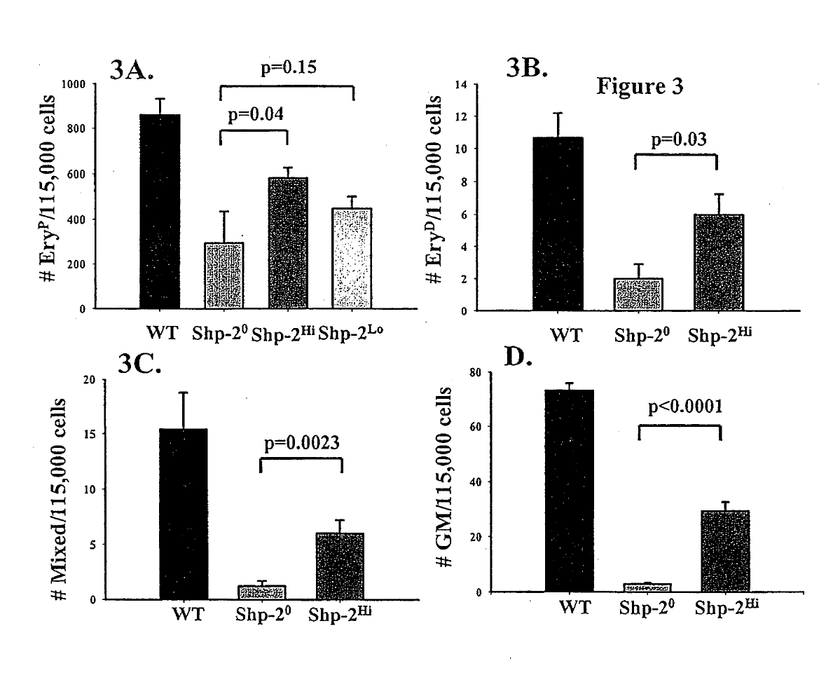

To evaluate this rescued phenotype more thoroughly, we collected 1° EBs on day 5 of differentiation and dissociated them into a single cell suspension and plated them into primitive erythroid progenitor assays. We observed that the negative control, Shp-2⁰ cell line, had a three- to four-fold lower capacity to differentiate into primitive erythroid progenitors compared to the wild-type cells (FIG. 3A). Upon reconstitution with wild-type Shp-2, differentiation into primitive erythroid cells was rescued in both the Shp-2^{Hi} and Shp-2^{Lo} cell lines, again in a dose-dependent manner. These results provided strong evidence that wild-type Shp-2 expression resulted in restoration of ES cell differentiation capacity and, more specifically, rescue of primitive erythropoiesis.

To examine definitive hematopoiesis, we next evaluated the capacity of ectopic WT Shp-2 expression to rescue definitive erythropoiesis and myelopoiesis. We found that the requirement of Shp-2 for definitive hematopoiesis is much more stringent than that of primitive hematopoiesis as the Shp-2^{Lo} cell line did not have increased definitive hematopoiesis or myelopoiesis compared to the Shp-2⁰ cell line in multiple experiments. However, definitive hematopoiesis was observed in the Shp-2^{Hi} cell line as demonstrated by increased numbers of definitive erythroid, mixed, and granulocyte-macrophage colonies compared to the Shp-2⁰ cell line (FIGS. 3B-D). These data are consistent with our previous observations that the requirement of Shp-2 for hematopoiesis is much more stringent than for other developmental programs (Qu, C. K. et al. 1998 Mol Cell Biol 18:6075-6082).

To evaluate hematopoietic differentiation of the Shp-2^{Hi} cell line at the molecular level, we examined the mRNA expression level of various hematopoietic-specific markers using semi-quantitative RT-PCR. All of the messages including, β-globin H1, β-globin major, a tyrosine kinase c-fms (the M-CSF (colony-stimulating factor) receptor), G-CSF R, and PU.1 (a hematopoietic-specific transcription factor) were expressed in each of the cell lines, although at a lower level in the Shp-2⁰ cell line compared to the WT and Shp-2^{Hi} cell lines. Conversely, the amount of the constitutively expressed gene, HRPT, was equal between all cell lines. These results demonstrate at a molecular level that ectopic expression of WT Shp-2 restores hematopoietic-specific gene expression, substantiating the functional data presented above.

Shp-2 Modulates LIF-Stimulated Stat3 Activity

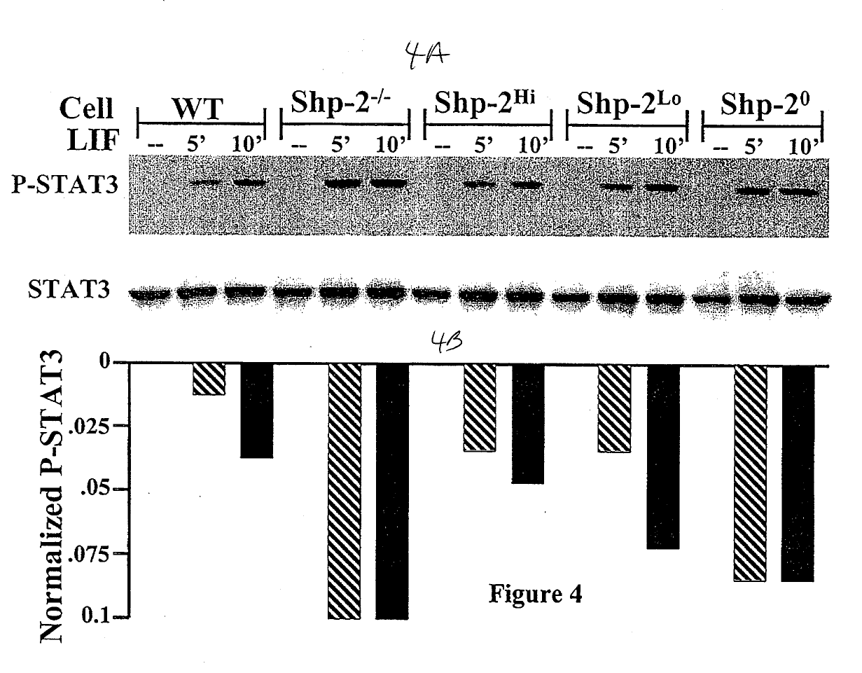

As the rescue cell lines, Shp-2^{Hi} and Shp-2^{Lo}, have been well characterized biologically, they serve as useful models to investigate the molecular mechanisms that underlie the observed defective phenotypes. The LIF-stimulated activation of Stat3 has been shown to be important in maintaining ES cells in an undifferentiated state (Matsuda, T. et al. 1999 EMBO J 18:4261-4269; Raz, R. et al. 1999 PNAS USA 96:2846-2851). Based on these previous studies and the observation that ES cells lacking functional Shp-2 have a severe defect in differentiation and are hypersensitive to LIF, Stat3 was proposed to be a potential target for Shp-2 modulation. Each of the cell lines was deprived of LIF stimulation in serum-free, LIF-free media for 6 hours followed by stimulation with 1000 U/mL LIF for 5 or 10 minutes. Clarified cell lysates were prepared and the amount of phospho-Stat3 was determined for each cell line. We found that the level of LIF-stimulated phospho-Stat3 was higher in the IC3 (Shp-2^{-/-}) ES cells compared to wild-type cells (FIG. 4). We observed that increasing wild-type Shp-2 expression corresponded to decreasing LIF-stimulated phospho-Stat3 levels in the Shp-2^{Hi}, Shp-2^{Lo}, and Shp-2⁰ cell lines in a dose-dependent manner, suggesting that Shp-2 downregulates the LIF-stimulated phospho-Stat3 pathway in ES cells.

Shp-2 Negatively Regulates ES Cell Self-Renewal and ES Cell Survival

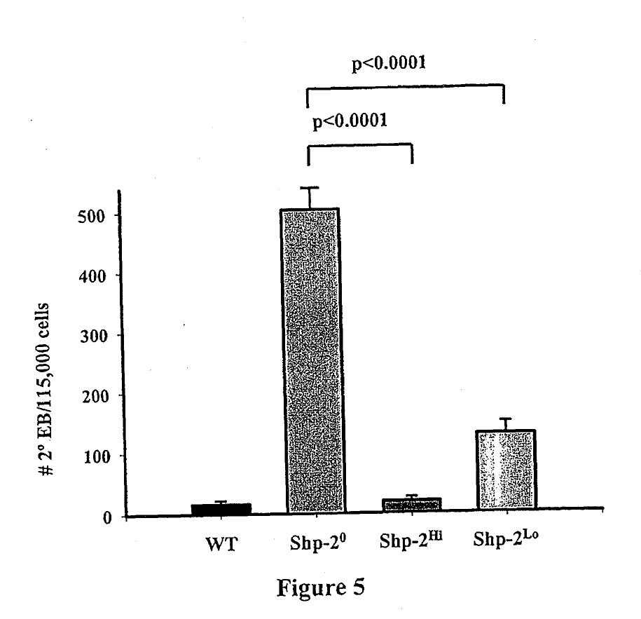

We next sought to investigate ES cell functions shown previously to be modulated by LIF and activated Stat3 such as self-renewal (Burdon, T. et al. 1999 Cells Tissues Organs 165:131-143; Niwa, H. et al. 1998 Genes Dev 12:2048-2060) and programmed cell death (Epling-Burnette, P. K. et al. 2001 J Clin Invest 107:351-362; Catlett-Falcone, R. et al. 1999 Immunity 10:105-115; Pesce, M. et al. 1993 Development 118:1089-1094). When 1° EBs are dissociated and replated in secondary culture, cells that fail to commit and differentiate in the primary culture yet retain pluripotency have the capacity to grow into new EBs, termed 2° EBs. The number of 2° EBs reflects self-renewal capacity (Qu, C. K. & Feng, G. S. 1998 Oncogene 17:433-439). To this end, we compared the number of 2° EBs derived from each of the ES cell lines. We observed that the Shp-2^{-/-} cells (as well as the parental Shp-2^{-/-} ES cells) had a dramatically higher number of 2° EBs compared to the wild-type cell line (FIG. 5). Upon reconstitution with wild-type Shp-2, the number of 2° EBs returned to nearly wild-type levels as seen with Shp-2^{Hi}. This phenotype was partially rescued as seen with Shp-2^{Lo} (FIG. 5). Again, the fact that this effect was observed in a dose-dependent manner provided solid evidence that the effect of Shp-2 restoration is a specific one.

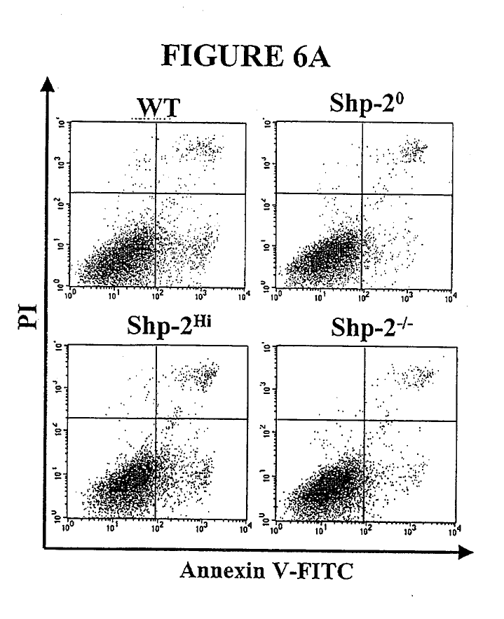

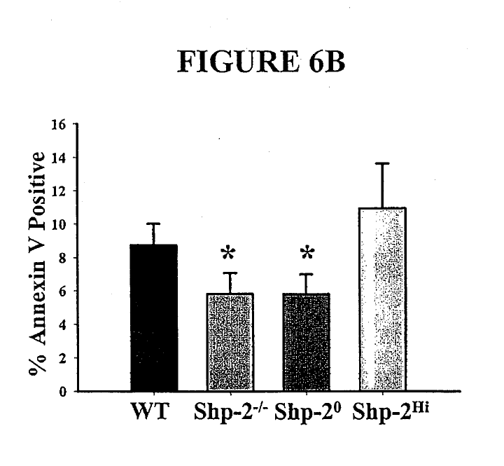

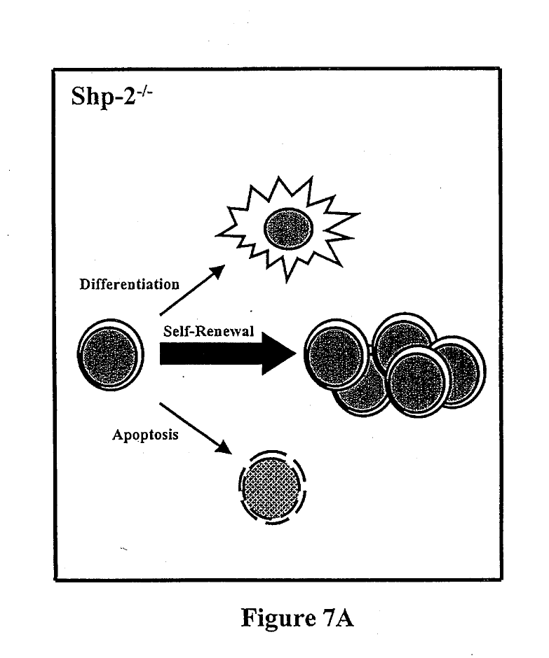

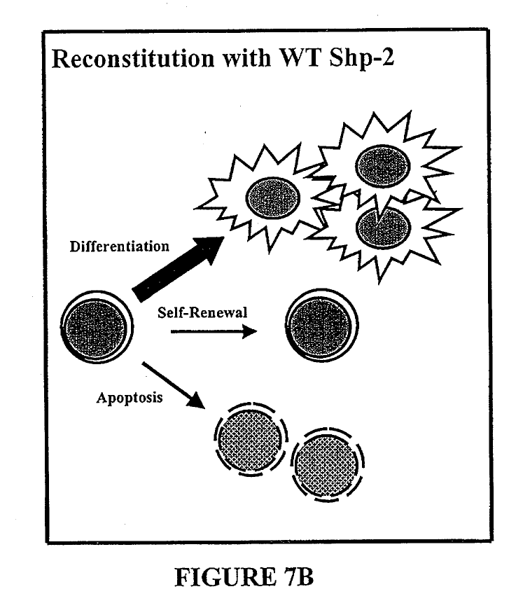

To investigate further the increase in 2° EBs seen with the Shp-2⁰ cells, we compared the level of programmed cell death (apoptosis) between various cell lines. As a cell undergoes apoptosis, loss of plasma membrane asymmetry is one of the earliest morphological changes. This loss of asymmetry occurs as the phospholipid phosphatidylserine move from an intra- to extra-cellular position on the plasma membrane. Extra-cellular phosphatidylserine is detected by staining with the fluorochrome-conjugated phospholipid-binding protein, annexin V (Vermes, I. et al. 1995 J Immunol Methods 184:39-51). Using this staining method, we observed that the Shp-2^{-/-} and Shp-2⁰ cells had a modest but significant decrease in apoptosis compared to the WT cells following continuous culture for 96 hours without change or supplementation of media (FIG. 6B). The level of apoptosis returned to WT levels upon reconstitution with WT Shp-2 as seen with the Shp-2^{Hi} cell line. These data suggest that absence of functional Shp-2 yields a survival advantage to ES cells. This survival advantage is likely contributing to the increased frequency of 2° EBs observed. However, as the increase in survival for the Shp-2^{-/-} and Shp-2⁰ cells was approximately 1.5-fold greater than the WT cells, whereas the increase in 2° EB frequency was from 10- to 30-fold greater (based on multiple experiments comparing the 2° EB frequency of the Shp-2⁰ and Shp-2^{-/-} cells to that of the WT cells), survival alone could not account for the increase in 2° EB frequency as observed in the Shp-2⁰ ES cells. It is likely that other LIF- and Stat3-regulated functions in addition to survival are enhanced resulting in the observed increase of ES cell self-renewal.

Shp-2^{-/-} ES cells, which have a decreased capacity to differentiate into hematopoietic progenitors and increased sensitivity to the cytokine LIF, provide an excellent model to study the role of Shp-2 in the molecular mechanisms that determine a stem cell's fate, such as commitment and differentiation, self-renewal, or programmed cell death. As a first step in elucidating the mechanism, we sought to reconstitute expression of wild-type Shp-2 in Shp-2^{-/-} ES cells. A plasmid containing the human β actin promoter was used to drive expression of the Shp-2 cDNA to generate stably transfected ES cell lines. The human β actin promoter was chosen as it has been used successfully by others to overexpress proteins in ES cells (Cheng, A. M. et al. 1998 Cell 95:793-803). We then selected clones that demonstrated reconstitution of Erk activity in response to LIF stimulation, as decreased Erk activity is a biochemical hallmark of Shp-2^{-/-} ES cells as well as Shp-2^{-/-} fibroblasts (Qu, C. K. et al. 1997 Mol Cell Biol 17:5499-5507; Shi, Z. Q. et al. 1998 J Biol Chem 273:4904-4908). Using these reconstituted cell types, we have shown that the wild-type phenotype is rescued in functional studies including colony differentiation upon LIF withdrawal, primary differentiation into hemoglobinized EBs, and differentiation into primitive and definitive hematopoietic cells, in a dose-dependent manner. These data unequivocally define a role of Shp-2 in mammalian hematopoiesis.

Based on the previous findings that LIF-stimulated phospho-Stat3 is necessary for ES cell pluripotency (Matsuda, T. et al. 1999 EMBO J 18:4261-4269; Raz, R. et al. 1999 PNAS USA 96:2846-2851) and self-renewal (Burdon, T. et al. 1999 Cells Tissues Organs 165:131-143; Niwa, H. et al. 1998 Genes Dev 12:2048-2060) and that Shp-2^{-/-} ES cells require lower concentrations of LIF for maintenance of an undifferentiated state (Qu, C. K. & Feng, G. S. 1998 Oncogene 17:433-439), we speculated that LIF-stimulated phospho-Stat3 would be greater in ES cells lacking functional Shp-2. We did indeed observe an increased level of LIF-stimulated phospho-Stat3 in the Shp-2^{-/-} and Shp-2⁰ compared to the WT cells. Upon reconstitution of the Shp-2^{-/-} ES cells with WT Shp-2, the level of LIF-stimulated phospho-Stat3 decreased in a dose-dependent manner, providing strong evidence that Shp-2 downregulates the LIF-stimulated phospho-Stat3 pathway in ES cells. We next evaluated known phospho-Stat3 mediated functions, such as self-renewal and apoptosis. We observed that ES cells lacking functional Shp-2 had increased self-renewal as evaluated in 2° EB assays, and this abnormality was corrected in a dose-dependent manner upon reconstitution with WT Shp-2. We also observed that ES cells lacking a functional Shp-2 had a higher level of survival when compared to the WT or Shp-2^{Hi} cell line. Based on these data, we propose that one of the crucial signaling mechanisms regulated by Shp-2 in ES cells is the LIF-stimulated level of phospho-Stat3, as the functional observations of decreased differentiation, increased self-renewal, and increased survival correlated well with the level of LIF-induced Stat3 activation. Even though the functional experiments of ES cell differentiation and in vitro hematopoiesis were performed in the absence of pharmacologic doses of LIF, we believe that the difference in LIF-stimulated phospho-Stat3 between the WT and Shp-2^{-/-} ES cells is operative at physiologic levels of LIF, as the hypersensitivity of the Shp-2^{-/-} ES cells originally was observed at 15 and 60 U/mL of LIF (Qu, C. K. & Feng, G. S. 1998 Oncogene 17:433-439).

We demonstrated the observed abnormalities of ES cell function due to lack of functional Shp-2 in FIG. 6A. Lack of functional Shp-2 caused ES cells to remain uncommitted and undifferentiated and to exhibit a lower level of apoptosis. The overall result was the maintenance of ES cells in an undifferentiated, replicative stem cell compartment with resulting increased self-renewal. The biochemical abnormalities demonstrated by the aberrantly functioning Shp-2^{-/-} ES cells included decreased LIF-stimulated phospho-Erk and increased LIF-stimulated phospho-Stat3. Our findings are consistent with the model generated by Burdon et al. who proposed that the balance between Stat3 activation, which promotes ES cell self-renewal, and Erk activation, which is dispensable for self-renewal and likely promotes differentiation, determines stem cell fate (Burdon, T. et al. 1999 Cells Tissues Organs 165:131-143; Burdon, T. et al. 1999 Dev Biol 210:30-43). Upon reconstitution with WT Shp-2 (FIG. 6B), all of these functional defects were normalized in conjunction with normalization of the LIF-stimulated Erk and Stat3 activation. Taken together, these results show that Shp-2 facilitates hematopoiesis by downregulating signals, in particular the LIF-stimulated phospho-Stat3 pathway, that promote ES cell self-renewal and survival.

The implications of Shp-2 regulation in the ES cell functions of differentiation, self-renewal, and survival are far-reaching. This invention shows that modulation of Shp-2 activity within embryonic, and likely hematopoietic, stem cells impact on the stem cell's capacity to remain undifferentiated with retention of repopulation ability upon culture in vitro.

### EXAMPLE 1

ES Cell Culture and Cell Lines

All ES cell lines were maintained in DMEM with 4.5 gm/L glucose, 6 mM glutamine, 1 mM sodium pyruvate, 0.1 mM non-essential amino acids, 55 μM β-mercaptoethanol, 15% ES cell-qualified maintenance fetal calf serum (FCS, Hyclone, Logan, Utah), and 1000 U/mL LIF (ESGRO, Gibco BRL or Peptrotech, Rocky Hill, N.J.) on gelatinized tissue culture plates. The Shp-2^{-/-} ES cell line, IC3, has been described previously (Qu, C. K. et al. 1997 Mol Cell Biol 17:5499-5507). The mammalian vector phβA-Shp-2, used to express the Shp-2 cDNA in IC3 cells, was prepared by replacing the CMV promoter of pcDNA3.1/hygro (Invitrogen, Carlsbad, Calif.) with approximately 3 kb of the human β-actin promoter from the vector pBAP (Gunning, P. et al. 1987 PNAS USA 84:4831-4835). The Shp-2 cDNA as previously described (Ohnishi, H. et al. 1996 J Biol Chem 271:25569-25574) was sequenced and subcloned into the multiple cloning site. Reconstituted cell lines were generated by mixing 5.6×10⁶ IC3 cells with 40 μg linearized phβA-Shp-2 followed by electroporation (240 V, 500 μF) and selection in 0.3 mg/mL hygromycin. All clones were screened for expression of wild-type Shp-2 by immunoblotting.

Colony Differentiation Assay

ES cell lines were cultured at a colony dilution (from 250 to 500 cells/mL) on gelatinized tissue culture plates for 6 to 8 days in ES cell maintenance media. The resulting colonies were washed with phosphate-buffered saline and cultured for an additional 48 hours in LIF-free media. The colonies were fixed and stained with Giemsa. Colonies were scored as differentiated when surrounded by flattened, fibroblast-like outgrowths.

ES Cell Differentiation into Embryoid Bodies

For primary differentiation assays, ES cells were plated in bacterial grade petri dishes at a concentration of 1000-2000 cells/mL in 0.9% methylcellulose-based differentiation media which included Iscove's modified Dulbecco's medium (IMDM), 2 mM glutamine, penicillin/streptomycin (100 U/mL/100 μg/mL), 5% PFHM-II (Gibco BRL), 200 mg/mL iron-saturated holo-transferrin (Sigma, St. Louis, Mo.), 5 mg/mL ascorbic acid, 450 μM monothioglycerol (Sigma, St. Louis, Mo.), and 15% differentiation FCS (StemCell Technologies, Vancouver, BC) and incubated for 8 to 10 days at 37° C. in 5% CO₂. EBs were viewed by light microscopy and scored for the presence or absence of hemoglobin. For the formation of EBs used for secondary assays, ES cells were plated either in liquid-based differentiation media for day 5 or 6 EBs or in methylcellulose-based differentiation media for day 10 EBs.

Secondary Plating Assays

At day 5 to 6 (for 2° EB or primitive erythroid assays) or day 10 (for definitive erythroid, mixed, and granulocyte/macrophage assays) of differentiation, 1° EBs were collected and digested with either 0.25% trypsin (5 minutes, 37° C.) or 0.2% collagenase in 20% FCS (30 minutes, 37° C.) followed by dissociation into a single cell suspension by passaging through a 20 G needle 2-10 times. Cell concentrations and viability were performed using trypan blue. For all secondary plating assays, cells were plated at 50,000 cells/mL in methylcellulose-based differentiation media with the addition of erythropoietin (5 U/mL), stem cell factor (100 ng/mL), and IL-3 (1 ng/mL) for definitive erythroid assays and of erythropoietin (5 U/mL), stem cell factor (100 ng/mL), IL-3 (1 ng/mL), Granulocyte-Macrophage Colony-Stimulating Factor (GM-CSF) (10 ng/mL), and Macrophage Colony-Stimulating Factor (M-CSF) (5 ng/mL) for mixed and granulocyte/macrophage assays. For the detection of primitive erythroid progenitors, cells were plated in methylcellulose-based differentiation media with erythropoietin (5 U/mL) and the substitution of 15% plasma derived serum (Animal Technologies, Antech, Tex.) for FCS. Secondary EBs, primitive, and definitive erythroid progenitors were scored on day 7 of culture. Mixed and granulocyte/macrophage progenitors were scored at day 10-12 of culture. All growth factors were from PeproTech, Rocky Hill, N.J.

Immunoblot Analysis and Antibodies

Control and LIF-stimulated cell lysates were prepared as previously described (Feng, G. S. et al. 1994 Oncogene 9:1545-1550). ES cells were cultured in serum-free, LIF-free maintenance media containing 0.5% bovine serum albumin (Sigma, St. Louis, Mo.) for six hours followed by stimulation for various times with 1000 U/mL LIF. Clarified total cell lysates were electrophoresed on a 10% polyacrylamide gel followed by transfer to a nitrocellulose membrane. To detect both wild-type and mutant Shp-2, anti-SH-PTP2C (C18) from Santa Cruz Biotechnology, Inc. (Santa Cruz, Calif.) was utilized. Anti-phospho-Stat3, anti-Stat3, anti-phospho-Erk, and anti-Erk were from New England Biolabs (Beverly, Mass.). Signals were detected by enhanced chemiluminescence and quantified by densitometry.

RT-PCR Analysis

Total cellular RNA was prepared from day ten 1° EBs using QIAamp Blood (Qiagen, Valencia, Calif.). First strand synthesis of cDNA was performed using poly dT primer and reverse transcriptase (SuperScript™, Invitrogen, Carlsbad, Calif.). Primer pairs for β-globin H1, β-globin major, the M-CSF receptor (c-fms), the G-CSF receptor (G-CSF R) and PU.1 were used with the synthesized cDNA as a template to perform PCR. Semi-quantitative analysis was achieved by allowing the PCR reaction to proceed for various cycle numbers. The housekeeping gene, hypoxanthine phosphoribosyltransferase (HPRT) was used as an internal control. PCR products were subjected to agarose gel electrophoresis and stained with ethidium bromide.

Apoptosis Assay

ES cell lines were plated at 500,000 cells per 3.5 cm gelatinized plate and cultured for 24 hours in standard ES cell media. The media was changed and cells were cultured for an additional 96 hours without change of or addition to the media. The cells were collected by trypsinization, stained with annexin V-FITC and propidium iodide (BD PharMingen, San Diego, Calif.), and analyzed by FACS analysis.

Statistical Analysis

Groups were compared using the two-tailed Students t Test.

### EXAMPLE 2

Hematopoictic cells in the form of bone marrow cells or blood cells are obtained from a patient. The stem cell culture is prepared by methods well known in the art. The cells in culture are then treated with an Shp-2 inhibitor to reversibly inhibit their differentiation, and to increase their self-renewal and survival. After such ex vivo expanding using an Shp-2 inhibitor of the present invention, the stem cell culture is transplanted into a patient to alleviate the symptoms of a disease, tissue/organ degeneration or trauma.

While the present invention has been described in some detail for purposes of clarity and understanding, one skilled in the art will appreciate that various changes in form and detail can be made without departing from the true scope of the invention. All patents, patent applications and publications referred to above are hereby incorporated by reference.

What is claimed is:

1. A method of identifying a compound as a modulator of Shp-2 activity, comprising:
cultivating a Shp-2-expressing stem cells or progenitor cells in the presence of said compound;
monitoring differentiation, self-renewal, or apoptosis rates of said stem cells or progenitor cells, and identifying changes in said rates, thereby identifying said compound as a modulator of Shp-2 activity.

2. The method of claim 1, wherein said modulator is selected from the group consisting of: peptides, proteins, antibodies, peptidomimetics, polynucleotides, and small molecules.

3. The method of claim 1, wherein said modulator inhibits differentiation of said stem cells or progenitor cells.

4. The method of claim 1, wherein said modulator enhances self-renewal of said stem cells or progenitor cells.

5. The method of claim 1, wherein said modulator stimulates differentiation of said stem cells or said progenitor cells.

6. A method of proliferating cells in an undifferentiated state, comprising contacting said proliferating cells with an inhibitor of Shp-2 activity.

7. The method of claim 6, wherein said contacting is performed in vitro.

8. The method of claim 6, wherein said contacting is performed in vivo.

9. The method of claim 6, wherein said cells are stem cells.

10. A method of inducing stem cell differentiation, comprising contacting said stem cells with an agonist of an Shp-2 activity.

11. The method of claim 10, wherein said contacting is performed in vitro.

12. The method of claim 10, wherein said contacting is performed in vivo.
